(19) United States
(12) Patent Application Publication
(10) Pub. No.: US 2016/0015813 A1
(43) Pub. Date: Jan. 21, 2016

(54) NOVEL AND SYNERGISTIC COMPOSITION OF LECITHIN AND LYSOLECITHIN FOR IMPROVING BIOAVAILABILITY AND SOLUBILITY OF HYDROPHOBIC COMPOUNDS AND EXTRACTS

(71) Applicant: LAILA PHARMACEUTICALS PVT. LTD., VIJAYAWADA (IN)

(72) Inventors: Ganga Raju Gokaraju, Vijayawada (IN); Rama Raju Gokaraju, Vijayawada (IN); Kiran Bhupathiraju, Vijayawada (IN); Trimurtulu Golakoti, Vijayawada (IN)

(21) Appl. No.: 14/739,274

(22) Filed: Jun. 15, 2015

(30) Foreign Application Priority Data
Jun. 16, 2014 (IN) 2625/CHE/2013

(51) Int. Cl. A61K 47/24; A61K 36/9066; A61K 31/4745; A61K 31/12

(52) U.S. Cl. CPC A61K 47/24; A61K 31/12; A61K 36/9066; A61K 31/4745

(57) ABSTRACT

The present invention discloses a composition(s) of hydrophobic plant molecule(s) and/or extract(s) with enhanced bioavailability comprising hydrophobic active and synergetic combination of lecithin and lysolecithin and to the process for preparation thereof.

### TECHNICAL FIELD OF THE INVENTION

The present invention relates to novel and synergistic composition(s) of natural emulsifiers and method(s) of solubilizing hydrophobic compound(s) and extract(s). These compositions are highly soluble and bioavailable, providing significant concentration of the active compound into the blood stream.

The present invention further discloses synergistic composition of lecithin and lysolecithin for solubilizing hydrophobic compound(s) and/or extract(s) for improving their solubility and bioavailability.

The present invention is also directed towards process of preparing such composition(s) containing hydrophobic compounds(s)/extract(s) by heat energy.

### BACKGROUND OF THE INVENTION

The traditional knowledge in India is vast with over 20,000 medicinal plants recorded. These plants were being used as herbal medicine over the years from around 2500 BC. Ayurveda, Siddha and Unani are some of the very old traditional systems of medicine, which documented the wide biodiversity of plants. Medicinal plants like Aswagandha, Amla, Brahmi, Guggul, Long pepper, Tulsi, Henna, Haridra, Neem and many more have been traditionally in use for treating various ailments.

Despite these uses of medicinal plants over the years there has been a lag to deliver a therapeutically efficacious drug/nutraceutical from a plant source. The drawback with herbal compounds or extracts is their poor water soluble nature. These compounds have poor systemic bioavailability in addition to their rapid metabolism and elimination from the body. As a result though these compounds are active in in-vitro, they fail to provide a similar effect in in-vivo conditions.

Curcumin is one such compound which has been investigated for years to develop it as a therapeutic compound. It is considered as the major constituent of the rhizome of Curcuma longa, a very common spice ingredient in Indian and oriental cuisine. Many pharmacological activities have been reported for curcumin including anti-oxidant, anti-inflammatory, anti-tumor, antiseptic and anti-amyloid properties. Commercially available curcuminoids product is optimized to 95% curcuminoids, consisting of curcumin, demethoxycurcumin and bis-demethoxycurcumin. Curcuminoids have been proved to be remarkably safe in animal studies and in many clinical evaluations even at high doses (up to 12 g/day). However, the major problem limiting the commercial exploitation of their therapeutic effects is their low bioavailability and their elimination from the body within 30 mins. Most of the hydrophobic plant compounds/extracts encounter same problem. Most of the phytochemicals such as curcumin and resveratrol has bioavailability less than 1%. Increasing the efficacy of these hydrophobic compounds in in-vivo conditions by increasing their systemic availability is challenging.

Similarly compounds like Coenzyme Q10, Boswellic acids, bis-o-demethyl curcumin, Resveratrol, Hypericin, Bacoside(s), Xanthorhizol, Luteolin, Genistein, Wogonin, Morin, Kaempferol and several other plant derived compounds and extracts are poorly bioavailable. These compounds fail to achieve sufficient concentration in blood stream to impart any therapeutic action.

Hydrophobic drugs like Paclitaxel, Amphotericin and many others failed due to improper formulation and thereby poor bioavailability, in spite of their significant therapeutic benefits. Due to the poor oral bioavailability many of these drugs are administered intravenously, which is not as per patient compliance.

Formulation development is a challenging task with several parameters to be considered right from choice of excipients, process conditions and several other requisites. Emulsifiers are being used as excipients in several formulations. Emulsifiers, a type of surfactants, are excipients commonly used for lowering the surface tension and for interaction with hydrophobic materials. Emulsifiers can be natural or synthetic with a specific hydrophilic—lipophilic balance. Surfactants are classified as anionic, cationic, amphoteric and non-ionic based on the polar head group. Choosing an ideal emulsifier will fulfill the essential prerequisites of a bioavailable formulation, such as effective dose, stability and sustained release of active compound from the formulation.

Natural emulsifiers like lecithin, guar gum, gum acacia, lysolecithin, honey, egg yolk, alkyl polyglycoside and many others have been used as food ingredients, in cosmetics and other applications. Being natural in origin, these substances are not expected to have any toxicity or side effects. However, there compatibility with the active compound has to be thoroughly evaluated, prior to their use in pharmaceutical or supplement industry.

Natural emulsifiers, though preferable, are not explored much in the industry as they do not have significant emulsifying properties similar to the synthetic emulsifiers. Synthetic emulsifiers are synthesized in order to improve their emulsifying properties, stability and physiochemical properties. Hence using natural emulsifiers to solubilize or emulsify hydrophobic compounds requires innovative process and parameters to obtain the desired effect.

Phytosomes and liposomes are some of the technologies developed to deliver curcuminoids and other active compounds into the blood stream thereby increasing their bioavailability. Liposomes are colloidal, vesicular structures made of phospholipid bilayers for the purpose of drug delivery. Polar drugs are dissolved in the aqueous core of the liposome, while nonpolar lipid-soluble drugs are dissolved in the bilayer.

Phytosomes are bilayered vesicle similar to liposomes, with few fundamental differences. The active principle in phytosome is anchored/complexed to the polar head of phospholipids, where the polar functionalities of the active principle interact via hydrogen bonds with the charged phosphate head of phospholipids.

From the prior art it is understood that, curcumin complexed with lecithin is prepared using solvent evaporation technique to deliver a bioavailable curcumin product in the form of phytosome. PCT/EP2007/001487 (W0200/101551), U.S. Ser. No. 13/186,176 (US20120244134) disclose aqueous dispersions using complex stabilizer comprising lecithin and non-phospholipid via homogenization for improved bioavailability. U.S. Ser. No. 11/867,347 (US20080145411) disclose a method for increasing absorbability of Coenzyme Q10 in the presence of lysolecithin and an oil and fat. EP19990912665 (EP1063898) discloses a method for increasing the absorption of carotenoids in humans and poultry by the use of lecithin and lysolecithin. PCT/EP2012/061635 (EP2720554) discloses oil-in-water emulsions comprising a phospholipid emulsifier.

The problem associated with these products is that many of the products though claim high bioavailability, they fail to deliver sufficient quantities of active compound into the blood stream. Also, the active ingredient in many of the product will be present as conjugate of Glucuronide or Sulphate in the systemic circulation, which are pharmacologically inactive and get eliminated from the body. Similarly the compositions contain several other components in addition to the emulsifier or are prepared by complex process such as solvent evaporation.

Pharmacokinetics of the drug is one important criterion in formulating efficacious composition. The time to reach maximum concentration (Tₘₐₓ), Maximum concentration (Cₘₐₓ), Area under the curve (AUC) and Half-life (T_{1/2}) are some of the important parameters to establish the systemic bioavailability of a particular drug/formulation. The higher AUC and higher t_{1/2} will reduce the dose levels and also achieve enhanced bioavailability leading to enhanced therapeutic efficacy.

Moreover the conventional methods and regular solubilization techniques are not efficient enough to solubilize high concentration of the hydrophobic compounds/extracts. Due to their lipophilic and hydrophobic nature, the choice of the right excipients, their combination and process of formulating such product is key to achieve the desired product.

Hence, the inventiveness of the present invention lies in arriving at such synergistic ratio of lecithin and lysolecithin to solubilize the hydrophobic active compounds(s)/extract(s) using a process involving subjecting the mixture to heat energy to provide solubilized, highly bioavailable, synergistic and efficacious compositions.

Accordingly the present invention aims to provide a novel synergistic composition with enhanced bioavailability and efficacy for potential use in the field of drugs, nutritional/dietary supplements for human and/or animal application.

### SUMMARY OF THE INVENTION

In an important aspect, the invention provides novel composition(s) comprising solubilized hydrophobic active compound(s)/extract(s) using natural emulsifier(s) for improved bioavailability.

In yet another aspect, the invention provides novel composition(s) containing synergistic combination of lecithin and lysolecithin as emulsifier phase to solubilize hydrophobic active compound(s)/extract(s) for improved bioavailability.

In yet another aspect, the invention provides the process of preparing such novel composition(s) containing hydrophobic active compounds(s)/extract(s) and natural emulsifier phase to achieve highly bioavailable composition(s).

In yet another aspect, the invention provides novel water soluble composition(s) containing hydrophobic active compound(s)/extract(s) in combination with emulsifier phase to achieve enhanced and long lasting efficacy at low dose and low cost.

In yet another aspect, the invention provides a water soluble composition(s) containing hydrophobic active compound(s)/extract(s) in combination with emulsifier phase for various therapeutic, preventative and general health supplement applications in animals and human beings.

In yet another aspect, the invention provides a water soluble composition(s) containing hydrophobic active compound(s)/extract(s) in combination with emulsifier phase for use as pharmaceutical/dietary or nutraceutical supplement/health supplement/OTC product/Ayurvedic (botanical) medicine.

In yet another aspect, the invention provides a water soluble composition(s) containing hydrophobic active compound(s)/extract(s) in combination with emulsifier phase either in liquid, semisolid or solid dosage form.

Various embodiments disclosed herein are directed to a composition for use in a therapeutic formulation, comprising a synergistic mixture of from about 5% to about 25% of lecithin; and from about 75% to about 95% of lysolecithin, based on the combined weight of said lecithin and said lysolecithin, in combination with a hydrophobic active ingredient. The synergistic mixture may be used in an amount of from about 50% to about 99.99% by weight, based on the combined weight of the synergistic mixture and the active ingredient. The hydrophobic active ingredient in an amount of from about 0.01 to 50% by weight, based on the combined weight of the synergistic mixture and the active ingredient.

In various embodiments, the active ingredient is a natural compound, a semi-synthetic compound, or a synthetic compound. The active ingredient may be a curcuminoid, a boswellic acid, berberine, resveratrol, hypericin, a bacoside, xanthorhizol, luteolin, Coenzyme Q10, pyrogallol, genistein, wogonin, morin, or kaempferol. In some embodiments, salts or derivatives of these active ingredients may be used.

Various embodiments disclosed herein are directed to a composition for use in a therapeutic formulation, comprising a synergistic mixture comprising lecithin and lysolecithin, in combination with a hydrophobic active ingredient selected from the group consisting of a curcuminoid, berberine, mixtures thereof, salts thereof, and derivatives thereof. When the active ingredient is a curcuminoid, the curcuminoid may be curcumin, demethoxycurcumin, bisdemethoxycurcumin, bis-o-demethylcurcumin, or a mixture thereof. In various embodiments, the synergistic lecithin/lysolecithin mixture is used in an amount of from about 50% to about 99.99% by weight; and the hydrophobic active ingredient is used in an amount of from about 0.01% to 50% by weight; based on the combined weight of the synergistic mixture and the active ingredient.

In various embodiments, the synergistic lecithin/lysolecithin mixture is used in an amount of from about 73% to about 90% by weight; and the hydrophobic active ingredient is used in an amount of from about 10% to 27% by weight; based on the combined weight of the synergistic mixture and the active ingredient.

Various embodiments disclosed herein are directed to a kit comprising a first dosage form and a second dosage form. The first dosage form contains a synergistic composition for use in a therapeutic formulation, comprising from about 5% to about 25% of lecithin and from about 75% to about 95% of lysolecithin, based on the combined weight of said lecithin and said lysolecithin. The second dosage form comprises a hydrophobic active ingredient, such as a curcuminoid or berberine. The kit may contain directions indicating that the first and second dosage forms are to be taken simultaneously.

Various embodiments disclosed herein relate to a composition for use in a therapeutic formulation, comprising a synergistic mixture comprising lecithin and lysolecithin, in combination with berberine, a salt thereof, or a derivative thereof. The mixture of lecithin and lysolecithin includes from about 5% to about 25% of lecithin; from about 75% to about 95% of lysolecithin, based on the combined weight of said lecithin and said lysolecithin. In various embodiments, the synergistic lecithin/lysolecithin mixture is used in an amount of from about 50% to about 99.99% by weight, or from about 73% to about 90% by weight, and the berberine active ingredient is used in an amount of from about 0.01% to 50% by weight; or from about 10% to 27% by weight, based on the combined weight of the synergistic mixture and the active ingredient.

Various embodiments disclosed herein relate to a kit comprising a first dosage form and a second dosage form. The first dosage form comprises a synergistic composition for use in a therapeutic formulation, comprising from about 5% to about 25% of lecithin and from about 75% to about 95% of lysolecithin, based on the combined weight of said lecithin and said lysolecithin. The second dosage form comprising a hydrophobic active ingredient, wherein the active ingredient is berberine, a salt thereof, or a derivative thereof.

Various embodiments disclosed herein relate to a process for formulating a berberine composition by:
preparing a lecithin mixture of from about 5% to about 25% of lecithin and from about 75% to about 95% of lysolecithin, based on the combined weight of lecithin and lysolecithin;
heating the lecithin mixture;
dissolving berberine in the lecithin mixture to produce an active mixture; and
cooling the active mixture to room temperature.

### BRIEF DESCRIPTION OF THE DRAWINGS

FIG. 1: TEM images of new Curcuminoid composition natural and Meriva

FIG. 2: Oral Bioavailability of new Curcuminoid compositions and Meriva

FIG. 3: Oral Bioavailability of new Curcuminoid powder formulation (LPCQNP-01054) and Meriva

FIG. 4: Oral Bioavailability of new Berberine formulation (1132001F3)

### DETAILED DESCRIPTION OF THE INVENTION

The invention will now be described in detail in connection with certain preferred and optional embodiments, so that various aspects thereof may be fully understood and appreciated.

The term ‘enzyme-modified lecithin’ as used herein alternately refers and means lysolecithin.

Emulsifiers, a type of surfactants, are excipients commonly used for lowering the surface tension and for interaction with hydrophobic materials. Lecithin and lysolecithin are natural emulsifiers present in most of the plant and animal tissues as an important structural component of cell membranes. The major component of lecithin is phospholipid and that of lysophospholipid is lysolecithin. Lysolecithin is produced from lecithin by removal of its terminal fatty acid radical by phospholipase A. Lecithin and lysolecithin have emulsifying, surfactant and lubricant properties. However, they both have limited use in formulations as they do not possess strong emulsifying properties on par to the synthetic emulsifiers. During the search for novel bio-enhancing formulations of curcuminoids, the inventors have found unexpectedly that higher amount of hydrophobic compound(s)/extract(s), such as curcumin or curcumin derivatives or extracts can be solubilized in lecithin or lysolecithin by using a process involving heating the mixture of hydrophobic compound(s)/extract(s) and lecithin or lysolecithin at elevated temperature (>100° C.), and the composition so obtained surprisingly shows enhanced bio-availability. The bioavailability obtained for the compositions containing curcuminoids mixture and lecithin or lysolecithin is significantly better compared to that obtained with curcumin alone. In addition, the bio-availability is also better compared to the similar products disclosed in the prior art, for example Meriva (PCT/EP2007/001487).

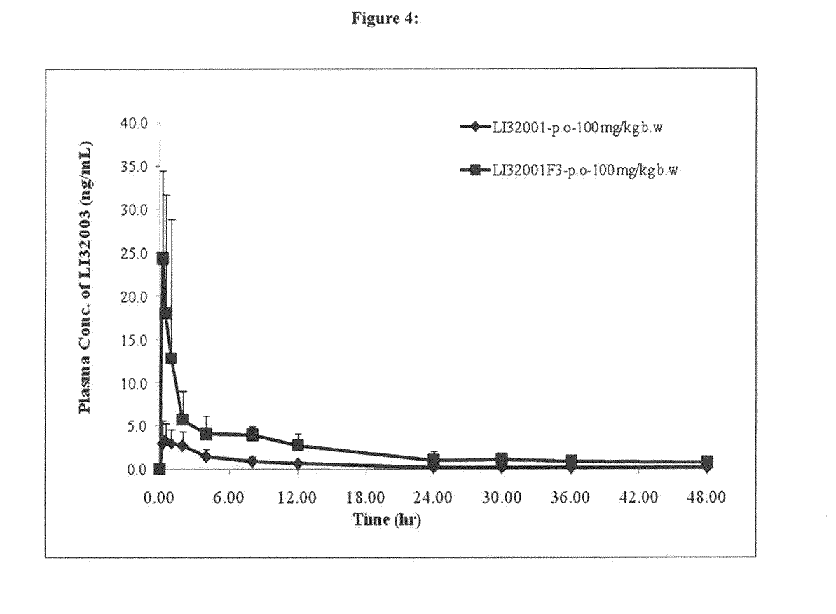

Further surprisingly, the inventors of the present invention have also found that combination of lecithin and lysolecithin when combined at ratios selected in a range could achieve superior bioavailability of phytochemical actives compared to those achieved by the individual ingredients lecithin and lysolecithin. For example, a composition/formulation (LPCQNO2-13) containing 10% of curcuminoids (curcuminoids 95%), 22.6% of lecithin and 67.4% of lysolecithin showed far better bioavailability (LPCQNO2-13; AUC 710.8±80.3) compared to the bioavailability obtained with composition containing lecithin (LPCQN05-13; AUC 330.62±18.8) alone and composition containing lysolecithin (LPCQN06-13; AUC 330.62±18.8) alone. Similarly, other Curcuminoid formulation (LPCQN08-13) and curcuminoids powder formulation (LPCQNP-01054) also showed significantly better bioavailability. A similar formulation was produced for another active called berberine. This formulation (LI32001F3) also showed significant improvement in oral bioavailability when compared to that obtained with unformulated Berberine compound (LI32001) as depicted in Table. 3 and FIG. 4. Beberine or its salts or the plant extracts containing berberine can be used for producing the formulations.

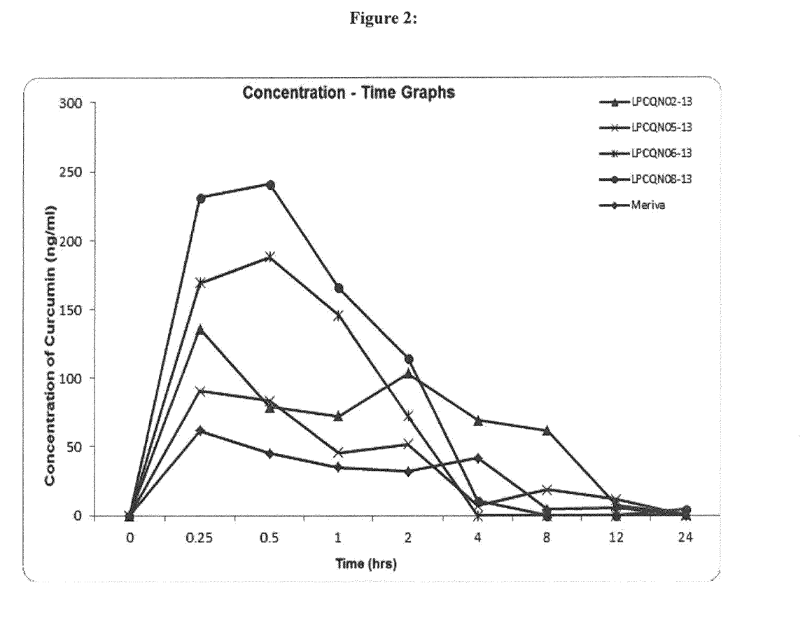

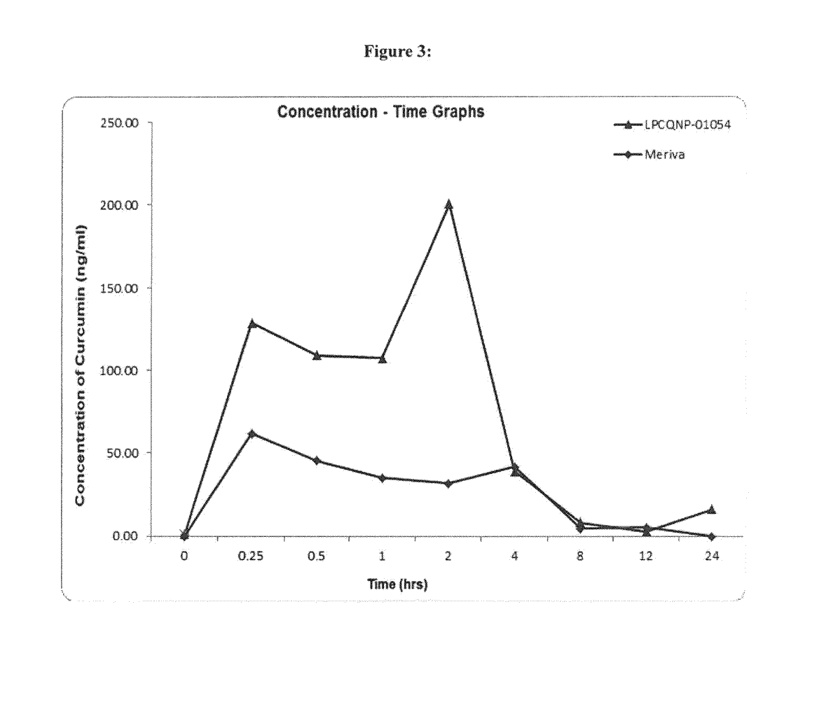

The details of the formulations used for bioavailability study is disclosed in Table 1, Example. 2, and Example. 3 and the typical process for the preparation of the formulations is disclosed in examples 4, 6 and 7 respectively for Curcuminoids formulations, Curcuminoids Powder formulation and Berberine formulation respectively. The bioavailability data is summarized in Tables. 2 and 3 for Curcuminoids formulations and Berberine formulation respectively. The results of comparative bioavailability study are also depicted in FIGS. 2, 3 and 4, respectively for Curcuminoids formulations, Curcuminoids powder formulation and Berberine formulation.

Based on the above it is obvious that the formulations containing both lecithin and lysolecithin shows synergistic enhancement of bioavailability of hydrophobic compound(s)/extract(s) such as curcuminoids. The bioavailability is also better compared to the marketed product, Meriva.

The use of lecithin and lysolecithin with hydrophobic compounds/extracts is well known in the prior art. However, the inventive step of the present invention lies in formulating a composition(s) containing a synergistic combination of lecithin and lysolecithin for solubilizing higher concentration of hydrophobic compound(s)/extract(s) for providing higher bioavailability and enhanced efficacy.

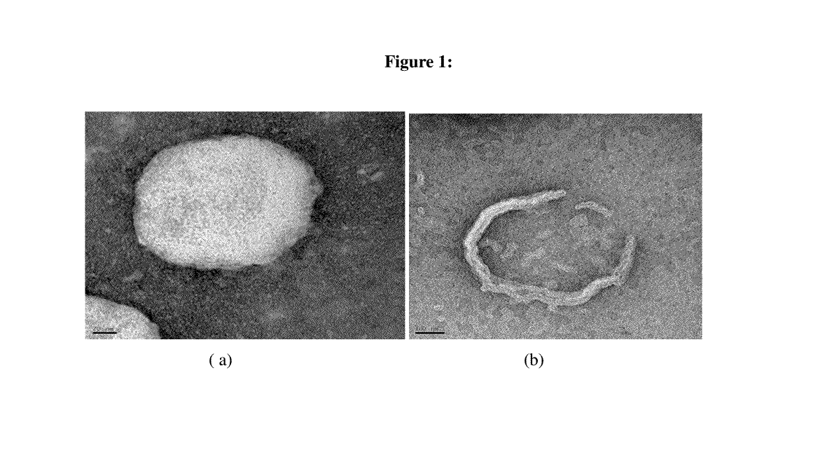

Phospholipid complexes of hydrophobic compounds are known to exist in bilayered structure in prior art. The preliminary assessment of the instant formulation under Transmission Electron Microscope indicated that the composition(s) of present invention form uni-layered micelles when added to water, whereas the known formulation Meriva exhibited bilayered structure as depicted in FIG. 1. The formulation of the present invention thus differs from the bilayered phospholipid complexes both in structure, solubility and pharmacokinetic properties. The uni-layered micelles are smaller in size than bilayered phospholipid complexes and are transported across the cell membrane through multiple ways, such as carrier mediated transport or may enter the cell by diffusion and/or through incorporation into the cell membrane. These uni-layered structures are spherical in shape and will be uniformly dispersed in an aqueous medium, with optimal surface tension.

Moreover the prior art documents teaches the compositions which comprise or use organic solvent, non-phospholipid components, fats, fatty acid esters, oil phase, aqueous phase and other components in addition to emulsifier(s). However the present invention is directed towards composition(s) containing hydrophobic compound(s)/extract(s) together with emulsifier phase (lecithin and lysolecithin) formulated by subjecting the mixture to heating to certain temperature.

Different embodiments of the present invention are as outlined below:

In a preferred embodiment, the invention describes composition(s) consisting of hydrophobic active compound(s)/extract(s) and emulsifier phase to deliver higher bioavailability.

In yet another embodiment, the invention describes a synergistic combination of lecithin and lysolecithin as emulsifier phase for use in said composition(s) consisting hydrophobic active compound(s)/extract(s).

The hydrophobic active compound(s)/extract(s) are of natural, semi-synthetic and/or synthetically derived.

The hydrophobic compound(s)/extract(s) are selected from but not restricted to Curcuma longa extract, Curcumin, Demethoxycurcumin, Bisdemethoxycurcumin, Bis-o-demethylcurcumin, and derivative of curcumin, Boswellia Serrata extract, Boswellic acids, Beta boswellic acid, keto beta boswellic acid, acetyl keto beta boswellic acid, Berberine, Resveratrol, Hypericin, Bacopa monneri extract, Bacoside A, Bacoside A3, Bacoside B, Xanthorhizol, Ginseng extract, Genistein, Gingko biloba, Pycnogenol, Coenzyme Q10, Luteolin, Kaempferol, Capsaicin, Rubia cordifolia extract, Lycopene, Pyrogallol, Lutein, Lawsennia iermis extract, Aloe vera extract, Beta carotene, Piperine and any other hydrophobic compounds/extracts.

The hydrophobic compound(s)/extract(s) in the said composition(s) can be present in an amount ranging from 0.01-50% using the method of the present invention and more preferably between 10-30%.

In yet another embodiment the invention describes the use of emulsifier phase in formulating said compositions containing hydrophobic compound(s)/extract(s). As used herein the term emulsifier(s) refers to substances which enhance the solubility of hydrophobic/lipophilic compounds/extracts.

According to the invention, the emulsifier phase contains a synergistic combination of lecithin and lysolecithin for solubilizing higher amount of hydrophobic compound(s)/extract(s) for providing enhanced bioavailability.

The composition(s) of the present invention entraps the hydrophobic compound(s)/extract(s) within a single layered Lecithin and Lysolecithin mixture micelle when added/mixed with aqueous phase. This entrapment offers bioprotection for the active compound(s) from hydrolytic and/or enzymatic degradation in the biological system.

The concentration of the Lecithin and Lysolecithin mixture in the said compositions ranges from 50 to 99.99% and more preferably 70-90%, where lecithin being upto 30%.

In yet another embodiment, the invention describes the process of preparing said compositions containing hydrophobic compound(s)/extract(s) and Lecithin and Lysolecithin mixture.

According to the inventive process, the process of preparing the said composition(s) involves the following steps:
a) weighing the required quantity of Lecithin and Lysolecithin;
b) Heating the Lecithin and Lysolecithin mixture;
c) weighing the required quantity of hydrophobic compound(s)/extract(s).
d) adding hydrophobic compound(s)/extract(s) to the heated Lecithin and Lysolecithin mixture batch wise over a period of time with continuous stirring and maintaining the temperature until complete solubilization; and
e) cooling the composition upon complete solubilization of the hydrophobic compound(s)/extract(s) in the Lecithin and Lysolecithin mixture the composition to room temperature.

Further the inventiveness of the present invention also lies in arriving at an ideal temperature to solubilize the hydrophobic compound(s)/extract(s) in the said emulsifier(s). Solubilization of hydrophobic compound(s)/extract(s) is possible only at temperature ranging from 90° C. to 140° C., more preferably between 115° C. to 125° C.

The present invention is directed to compositions and method of preparing the said compositions to improve the bioavailability for use in humans and/or animals as drug and/or dietary/nutritional supplement/OTC products/health supplements/ayurvedic (botanical) medicine.

In yet another embodiment, the invention is directed to composition(s) of hydrophobic compound(s)/extracts having anti-inflammatory, anti-allergic, anti-oxidant, memory enhancing, anti-obese, neuro-protective, anti-diabetic, anti-cancerous, cardio protective, eye protective and anti-microbial activities.

The composition(s) as disclosed in the present invention are administered as oral, nasal, anal, topical, vaginal, ocular, or buccal dosage forms.

In yet another aspect, the invention provides composition(s) containing hydrophobic active compound(s)/extract(s) in combination with emulsifier phase either in liquid, semisolid or solid dosage form.

In yet another embodiment, the present invention discloses composition(s) in a free flowing solid powder form, which is obtained by subjecting the liquid composition(s) to techniques not limited to encapsulation, nanospray drying, thin layer drying, freeze drying, using carriers like Microcrystalline cellulose, Precipitated Silica, Fujicalin, Nucelin, Mannitol, Hydroxypropyl Methylcellulose, Arbocel, Silica and cellulose derivatives.

In yet another embodiment, the present invention discloses composition(s) in a semi solid gel, lotion or cream form, which is obtained by formulating the liquid composition(s) with suitable polymers not limited to Hydroxypropyl Methylcellulose, Isopropyl myristate, Collagen, Glycerol, Cetyl alcohol, Sterates of magnesium, Zinc, Calcium and Carbopol.

The composition(s) of the present invention further are effective in delivering high concentrations of the active compound in to blood stream.

In yet another embodiment, the invention is directed to composition(s) of hydrophobic compound(s)/extract(s) for the treatment and/or prevention of inflammation, osteoarthritis, allergy, obesity, neuro-degenerative disorders, diabetes, cancer, cardio vascular disorders, microbial disorders and ocular diseases.

In yet another embodiment, the invention is directed to composition(s) of hydrophobic compound(s)/extract(s) which can be administered as pharmaceuticals/nutraceuticals/ayurvedic/dietical compositions to the subject in need thereof.

Various formulations were developed using one or combination of hydrophobic compound(s)/extracts along with single or combination of emulsifiers which are exemplified herein. Having described the invention with reference to certain preferred embodiments, other embodiments will become apparent to one skilled in the art from consideration of the specification.

The invention is further defined by reference to the following examples describing in detail the methods of preparation and use of the invention. It will be apparent to those skilled in the art that many modifications, both to materials and methods, may be practiced without departing from the scope of the invention.

### Example 1

### Composition of Curcuminoid Formulations (Liquid/Semi Solid/Paste)

| | | | Emulsifier Phase | |
|---|---|---|---|---|
| | Hydrophobic | Hydrophobic | Lecithin | Lysolecithin |
| S. No. | compound/extract | Active (gm) | (gm) | (gm) |
| 1 | Curcuminoids 95% | 11.5 | 5 | 83.5 |
| 2 | Curcuminoids 95% | 21.5 | 5 | 73.5 |
| 3 | Curcuminoids 95% | 22 | 10 | 68 |
| 4 | Curcuminoids 95% | 11 | 22 | 67 |

### Example 2

### Composition of Curcuminoid Powder Formulation (LPCQNP-01054)

| S. No | Ingredients | Quantity (g)/1000 g |
|---|---|---|
| 1 | Curcuminoids extract | 149.00 |
| 2 | Lecithin | 45.00 |
| 3 | Enzyme modified Lecithin | 302.85 |
| 4 | Lecithin Powder | 50.00 |
| 5 | Tocopherol | 0.90 |
| 6 | Sodium chloride | 2.25 |
| 7 | Xanthan gum | 10.00 |
| 8 | Micro Crystalline Cellulose (MCC) | 430.00 |
| 9 | Syloid | 10.00 |

### Example 3

### Composition of Berberine Formulation (LI32001F3)

| S. No | Ingredient | Quantity (g)/100 g |
|---|---|---|
| 1 | Berberine (LI32001) | 11.00 |
| 2 | Lecithin | 5.00 |
| 3 | Lecithin Powder | 5.00 |
| 4 | Enzyme modified Lecithin | 33.65 |
| 5 | NaCl | 0.25 |
| 6 | Tocopherol | 0.1 |
| 7 | MCC | 43.01 |
| 8 | Syloid | 1.99 |

### Example 4

### Process of Preparing 11.5% Curcuminoids Formulation

1. Emulsifiers Lecithin (5 g), Lysolecithin (83.5 g) were accurately weighed, mixed and heated to 120° C.
2. To the preheated emulsifier mixture, curcuminoids (11.5 g) was added and stirred continuously till the curcuminoids are completely dissolved.
3. On complete solubilization of the curcuminoids, the mixture is cooled to room temperature to obtain the final formulation.
   The obtained formulation is packed in suitable container.

### Example 5

### Process of Preparing 21.5% Curcuminoids Formulation

1. Emulsifiers (5 g) Lecithin, (73.5 g) Lysolecithin were accurately weighed and heated to 120° C.
2. To the preheated emulsifier mixture, curcuminoids (21.5 g) was added and stirred continuously till the curcuminoids are completely dissolved.
3. On complete solubilization of curcuminoids, the mixture is cooled to room temperature to obtain the final formulation.
   The obtained formulation is packed in suitable container.

### Example 6

### Process of Preparing Curcuminoid Powder Formulation (LPCQNP-01054)

1. Weighed required quantities of lecithin, enzyme modified lecithin, sodium chloride and Tocopherol into round bottom flask (Example. 2)
2. Heated the above contents to 130° C.
3. The curcuminoids were added to the above mixture at 130° C. with continuous stirring
4. Maintained the temperature between 138° C.-140° C. with continuous stirring for 2 hours 20 minutes
5. Allowed the above mixture to cool down to 80° C. and mixed weighed quantity of xanthan gum and lecithin powder
6. Allowed the mixture to cool down to room temperature, and blended with MCC followed by addition of Syloid.
7. The end product is a free flowing powder.

### Example 7

### Process of Preparing Berberine Formulation (LI32001F3)

1. Weighed required quantities of lecithin, enzyme modified lecithin, sodium chloride and Tocopherol into round bottom flask (Example. 3)
2. Heated the above contents to 130° C.
3. Berberine was added to the above mixture at 130° C. with continuous stirring
4. Maintained the temperature between 138° C.-140° C. with continuous stirring for 2 hours 20 minutes
5. Allowed the above mixture to cool down to 80° C. and mixed weighed quantity of lecithin powder
6. Allowed the mixture to cool down to room temperature and blended with MCC followed by addition of Syloid
7. The end product is a free flowing powder.

### Example 8

### Transmission Electron Microscopy of Curcuminoid Formulation and Meriva Sample

1. Curcuminoid formulation
2. Meriva (Batch No FG-6558)

Sample Preparation: Sample was diluted in double distilled water in the ratio of 1:10000 (sample: water), vortexed well and allowed for standing for 5 mins. Supernatant was taken up for TEM studies.

Grid Preparation:

Copper grids were used for the study. Grid was neutralized before the initiation of sample loading.

Sample Loading:

10 μl of prepared sample was loaded on to the charge neutralized grid. Sample was allowed to settle on to the grid for 10 mins. Excess sample was blotted off carefully and allowed for drying for 15 mins.

Staining:

10 μl of 1% uranyl acetate was added and left undisturbed for 10 seconds. Excess dye was blotted out and allowed to dry for 15 mins.

Stained grid is then observed using TEM for presence of micelles.

Results:

Curcuminoid formulation of the present invention, when diluted with water and visualized under transmission electron microscope showed a single layered spherical structure encapsulating curcuminoids at the core of the structure. On the other hand Meriva was clearly visualized as a double layered structure, which is known to be the inherent property of phytosome or liposome.

### Example 9

### Oral Bioavailability Study

Pharmacokinetics (PK) is a fundamental scientific discipline that underpins applied therapeutics. Drugs with poor PK are reported to be poorly absorbed into the biological system and hence are therapeutically inefficient.

The study was conducted to evaluate the bioavailability of orally administered curcuminoid compositions (Table 1 and Example. 2), formulated using a synergistic combination of lecithin and lysolecithin in Sprague Dawley rats. Compositions formulated with lecithin or lysolecithin alone were also tested along with Meriva (Batch No FG-6558). These formulations were prepared using similar procedure described in examples 2 and 3. Total of twelve (12) animals were divided into four groups having three animals in each group. The test substances were administered orally to the animals via oral gavage once at 125 mg/Kg body weight active ingredient equivalent to Active ingredient. Pre-dose or 0th time blood samples were collected from all the animals before dosing the test substance, followed by blood sampling at 0.15, 0.5, 1, 1.5, 2, 4, 8, 12 and 24 hrs after dosing the test substance by sinus orbital plexus under anesthesia. Serum was separated from blood by centrifugation and was subjected to LC-MS/MS analysis for estimation of curcumin concentration in serum samples.

The similar protocol was used for the evaluation of oral bio-availability of berberine formulation (Example. 3) in comparison with the unformulated berberine and for estimation of berberine concentration in serum samples. The berberine compound and its formulations were supplemented to the animals at a dose equivalent to 100 mg/kg bodyweight of active Berberine.

Bioavailability data of all the tested Curcuminoids formulations showing the synergistic effect of Lecithin and Lysolecithin is shown in Table. 2 and FIGS. 2 & 3 along with bioavailability data for Meriva. Bioavailability data for Berberine composition in comparison to unformulated Berberine is shown in Table. 3 and FIG. 4.

TABLE 1
Details of tested Curcuminoid formulations

| Curcuminoid | Lecithin | Lysolecithin | Curcuminoids | Total |
|---|---|---|---|---|
| Formulations | (g) | (g) | (g) | (g) |
| LPCQN-02-13 | 22.6 | 67.4 | 10 | 100 |
| LPCQN05-13 | 89 | 0 | 11 | 100 |
| LPCQN06-13 | 0 | 89 | 11 | 100 |
| LPCQN08-13 | 5 | 83 | 12 | 100 |

| | |
|---|---|
| Meriva | Batch No FG-6558 |

The results obtained confirmed superior bioavailability of curcuminoid formulation comprising combination of lecithin and lysolecithin. This synergistic combination was found to be superior compared to compositions formulated with lecithin and lysolecithin alone and Meriva. Minor quantities of curcumin were detected at 0th time in tested compositions which might be due to contamination during manual handling.

The summary of PK results is tabulated in Table 2. The study confirmed the superior bioavailability of curcuminoid composition formulated using synergistic combination of lecithin and lysolecithin, which can be correlated to its enhanced efficacy.

TABLE 2
Pharmacokinetic parameters of tested Curcuminoids Formulations

| | No. | | | |
|---|---|---|---|---|
| | of | Dosage | AUCₗₐₛₜ | |
| | Ani- | (mg/kg | (0-24 h) | Cₘₐₓ |
| Products | mals | BW) | (ng/mL*hr) | (ng/mL) |
| LPCQN02- | 3 | 125 | 710.8 ± 80.3 | 136.2 ± 10.6 |
| 13 | | | | |
| LPCQN05- | 3 | 125 | 352.89 ± 111.4 | 98.43 ± 16.8 |
| 13 | | | | |
| LPCQN06- | 3 | 125 | 330.62 ± 18.8 | 197.28 ± 23.9 |
| 13 | | | | |
| LPCQN08- | 3 | 125 | 507.20 ± 96.4 | 260.91 ± 17.7 |
| 13 | | | | |
| LPCQNP- | 3 | 125 | 658.17 ± 396.33 | 274.76 ± 229.56 |
| 01054 | | | | |
| Meriva | 3 | 125 | 291.01 ± 33.2 | 61.86 ± 7.7 |

TABLE 3
Pharmacokinetic parameters of tested
Berberine Formulation (LI32001F3)

| | No. | | | |
|---|---|---|---|---|
| | of | Dosage | AUCₗₐₛₜ | |
| | Ani- | (mg/kg | (0-24 h) | Cₘₐₓ |
| Products | mals | BW) | (ng/mL*hr) | (ng/mL) |
| LI32001F3 | 6 | 100 | 102.86 ± 17.16 | 26.56 ± 13.02 |
| LI32001 | 6 | 100 | 23.66 ± 7.99 | 4.20 ± 1.85 |

Following a single oral administration of LI32001 and LI32001F3, Cₘₐₓ of LI32003 in LI32001 and LI32001F3 formulations was 4.20±1.85 and 26.56±13.02 ng/mL, respectively. Oral exposures (AUCₗₐₛₜ) of LI32003 in LI32001 and LI32001F3 formulations were 23.66±7.99 and 102.86±17.16 hr*ng/mL, respectively (Table 3). LI32001F3 formulation showed 6.31-fold and 3.35-fold higher Cₘₐₓ and exposure (AUCₗₐₛₜ) of LI32003 as compared to LI32001 formulation after per oral administration of equivalent dose of the active ingredient respectively (FIG. 4).

We claim:

1. A composition for use in a therapeutic formulation, comprising;
a synergistic mixture comprising from about 5% to about 25% of lecithin; and from about 75% to about 95% of lysolecithin, based on the combined weight of said lecithin and said lysolecithin; in combination with:
a hydrophobic active ingredient.

2. The composition of claim 1, comprising:
said synergistic mixture in an amount of from about 50% to about 99.99% by weight; and
said hydrophobic active ingredient in an amount of from about 0.01 to 50% by weight; based on the combined weight of said synergistic mixture and said active ingredient.

3. The composition of claim 3, wherein:
the active ingredient is at least one compound selected from the group consisting of a curcuminoid, a boswellic acid, resveratrol, hypericin, a bacoside, xanthorhizol, luteolin, Coenzyme Q10, pyrogallol, genistein, wogonin, morin, kaempferol, salts thereof, and derivatives thereof.

4. The dosage form of claim 2, wherein the active ingredient is a natural compound, a semi-synthetic compound, or a synthetic compound.

5. A composition for use in a therapeutic formulation, comprising;
a synergistic mixture comprising from about 5% to about 25% of lecithin; and from about 75% to about 95% of lysolecithin, based on the combined weight of said lecithin and said lysolecithin; in combination with:
a hydrophobic active ingredient, wherein the active ingredient is berberine, a salt thereof, or a derivative thereof.

6. A composition of claim 1, comprising;
said synergistic mixture comprising from about 5% to about 25% of lecithin; and from about 75% to about 95% of lysolecithin, based on the combined weight of said lecithin and said lysolecithin; in combination with:
said hydrophobic active ingredient, wherein said hydrophobic active ingredient is a curcuminoid, a salt thereof, or a derivative thereof.

7. The composition of claim 6, wherein the curcuminoid is curcumin, demethoxycurcumin, bisdemethoxycurcumin, bis-o-demethylcurcumin, or a mixture thereof.

8. The composition of claim 5, comprising:
said synergistic mixture in an amount of from about 50% to about 99.99% by weight; and
said hydrophobic active ingredient in an amount of from about 0.01% to 50% by weight; based on the combined weight of said synergistic mixture and said active ingredient.

9. The composition of claim 6, comprising:
said synergistic mixture in an amount of from about 50% to about 99.99% by weight; and
said hydrophobic active ingredient in an amount of from about 0.01% to 50% by weight; based on the combined weight of said synergistic mixture and said active ingredient.

10. The composition of claim 5, comprising:
said synergistic mixture in an amount of from about 73% to about 90% by weight; and
said hydrophobic active ingredient in an amount of from about 10% to 27% by weight; based on the combined weight of said synergistic mixture and said active ingredient.

11. The composition of claim 6, comprising:
said synergistic mixture in an amount of from about 73% to about 90% by weight; and
said hydrophobic active ingredient in an amount of from about 10% to 27% by weight; based on the combined weight of said synergistic mixture and said active ingredient.

12. The composition of claim 1, wherein the active ingredient is an extract.

13. The composition of claim 12, wherein the active ingredient is an extract of Curcuma longa, Ginseng, Ginkgo biloba, Garcinia mangostana, Ocimum basilicum, Zingiber officinale, Tribulus terrestris, Sphaeranthus indicus, Annona Squamosa, Moringa oleifera, Murraya koenigii, or a mixture thereof.

14. The composition of claim 1, wherein said composition further comprises at least one pharmaceutically acceptable excipient, said composition being formulated as a liquid, semisolid or solid dosage form.

15. The composition of claim 14, wherein said dosage form is a solid dosage form, and said pharmaceutically acceptable excipient is Microcrystalline cellulose, Precipitated Silica, calcium phosphate, Mannitol, Hydroxypropyl Methylcellulose, Silica, or a mixture thereof.

16. The composition of claim 14, wherein said dosage form is a semisolid dosage form, and said pharmaceutically acceptable excipient is Hydroxypropyl Methylcellulose, Isopropyl myristate, Collagen, Glycerol, Cetyl alcohol, a metal salt of stearatic acid, and a polymer of acrylic acid.

17. A kit comprising a first dosage form and a second dosage form;
said first dosage form comprising a synergistic composition for use in a therapeutic formulation, comprising from about 5% to about 25% of lecithin and from about 75% to about 95% of lysolecithin, based on the combined weight of said lecithin and said lysolecithin; and
said second dosage form comprising a hydrophobic active ingredient.

18. The kit of claim 17, wherein the active ingredient is a curcuminoid.

19. The kit of claim 18, wherein the curcuminoid is curcumin, demethoxycurcumin, bisdemethoxycurcumin, bis-O-demethylcurcumin, or a mixture thereof.

20. A kit comprising a first dosage form and a second dosage form;
said first dosage form comprising a synergistic composition for use in a therapeutic formulation, comprising from about 5% to about 25% of lecithin and from about 75% to about 95% of lysolecithin, based on the combined weight of said lecithin and said lysolecithin; and
said second dosage form comprising a hydrophobic active ingredient,
wherein the active ingredient is berberine, a salt thereof, or a derivative thereof.

21. A process for formulating the composition of claim 1, comprising;
preparing a lecithin mixture of from about 5% to about 25% of lecithin and from about 75% to about 95% of lysolecithin, based on the combined weight of said lecithin and said lysolecithin;
heating said lecithin mixture;
dissolving said hydrophobic active ingredient in said lecithin mixture to produce an active mixture; and
cooling the active mixture to room temperature.

22. The process according to claim 21, wherein:
in step (b), said lecithin mixture is heated to a temperature of about 90° C. to about 140° C.

23. The process according to claim 21, wherein:
in step (c), said hydrophobic active ingredient is dissolved in said lecithin mixture at a temperature of about 90° C. to about 140° C.

24. The process according to claim 21, wherein the active ingredient is a curcuminoid.

25. The process according to claim 24, wherein the curcuminoid is curcumin, demethoxycurcumin, bisdemethoxycurcumin, bis-O-demethylcurcumin, or a mixture thereof.

26. A process for formulating the composition of claim 5, comprising;
preparing a lecithin mixture of from about 5% to about 25% of lecithin and from about 75% to about 95% of lysolecithin, based on the combined weight of said lecithin and said lysolecithin;
heating said lecithin mixture;
dissolving said berberine in said lecithin mixture to produce an active mixture; and
cooling the active mixture to room temperature.
